(19) United States
(12) Patent Application Publication
(10) Pub. No.: US 2015/0361388 A1
(43) Pub. Date: Dec. 17, 2015

(54) ULTRASONIC APPARATUS AND METHOD FOR CELL RELEASE

(71) Applicants: Gregory Roger MARTIN, Acton, ME (US); Allison Jean TANNER, Portsmouth, NH (US); CORNING INCORPORATED, Corning, NY (US)

(72) Inventors: Gregory Roger Martin, Acton, ME (US); Allison Jean Tanner, Portsmouth, NH (US)

(21) Appl. No.: 14/768,272

(22) PCT Filed: Feb. 26, 2014
(86) PCT No.: PCT/US2014/018481

§ 371 Date: Aug. 17, 2015

Related U.S. Application Data
(60) Provisional application No. 61/769,803, filed on Feb. 27, 2013.

(51) Int. Cl. C12M 1/26; C12N 13/00; C12M 1/32; C12M 1/24; C12M 1/22

(52) U.S. Cl. CPC C12M 33/00; C12M 23/08; C12M 23/10; C12M 23/12; C12N 13/00

(57) ABSTRACT

An apparatus (100) for cell release from a cell culture article (101), including: an ultrasonic source (105); a stage comprising a base (112) and a raised portion (114) that defines a cell culture article confinement area; and a mechanism for relative scan motion between the ultrasonic source and the cell culture article confined to the stage. Also disclosed is a method for cell release and harvesting from a cell culture article (101), including: securing a cell culture article in the disclosed cell release apparatus; and accomplishing relative motion scanning between the energized ultrasonic source and the cell culture article for a time, as defined herein.

This application claims the benefit of priority to U.S. Application No. 61/769,803 filed on Feb. 27, 2013 the content of which is incorporated herein by reference in its entirety.

The entire disclosure of any publication or patent document mentioned herein is incorporated by reference.

### BACKGROUND

The disclosure generally relates to an ultrasonic apparatus and a method for release of adherent particles, such as biological cell release, from the surface of a vessel.

### SUMMARY

The present disclosure provides an apparatus and a method that uses relative motion of an ultrasonic energy source to release or remove adherent particles, such as biological cells, from the surface of a vessel, such as a biological cell culture vessel.

### BRIEF DESCRIPTION OF DRAWINGS

In embodiments of the disclosure:

FIGS. 1A and 1B shows, respectively, top and side views of an exemplary apparatus having a movable ultrasonic source supported on a race structure adjacent to a retained and supported work piece such as a cell culture vessel.

FIG. 2 shows a top view of an alternative configuration of the apparatus of FIG. 1 having a movable structure and a stationary ultrasonic horn.

FIGS. 3A to 3C show images of useful exemplary alternative cell culture vessel types nested in the disclosed apparatus.

FIGS. 4A to 4D show an exemplary top view imaging sequence obtained from scanning the length of a stationary cell culture vessel (101) with a movable 20 kHz ultrasonic horn (105).

FIGS. 5A and 5B show, respectively, examples of actual and modeled cell release patterns.

FIGS. 6A and 6C show, respectively, examples images of modal patterns visualized with crystal violet stain.

FIG. 7 illustrates variation of parameters can provide improved and maximized yields of cell release.

FIG. 8 shows CHO K1 Cell yield on UPCELL™ (Nunc) with and without use of the disclosed apparatus and method for cell release.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

In embodiments, the disclosed apparatus, and the disclosed method of making and using the apparatus provide one or more advantageous features or aspects, including for example as discussed below. Features or aspects recited in any of the claims are generally applicable to all facets of the invention. Any recited single or multiple feature or aspect in any one claim can be combined or permuted with any other recited feature or aspect in any other claim or claims.

### DEFINITIONS

“Adherent,” “adhere,” or like terms refer to, for example, particles, such as biological cells, that are alive or lifeless, that are connected to, or associated with a surface, especially by physical contact.

“Include,” “includes,” or like terms means encompassing but not limited to, that is, inclusive and not exclusive.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. The claims appended hereto include equivalents of these “about” quantities.

“Optional” or “optionally” means that the subsequently described event or circumstance can or cannot occur, and that the description includes instances where the event or circumstance occurs and instances where it does not.

“Consisting essentially of” in embodiments can refer to, for example:

an apparatus having: an ultrasonic source; a stage comprising a base having a raised portion (e.g., a fence) that defines a cell culture article confinement area; and a mechanism for relative scan motion between the ultrasonic source and the cell culture article confined to the stage, such as the ultrasonic source scanning the length of the cell culture article; and

a method of using the apparatus including: securing a cell culture article in the disclosed cell release apparatus, such as placing the article on the stage and in contract with the raised portion; and accomplishing relative motion scanning between the ultrasonic source and the cell culture article, such as the ultrasonic source scanning the length of the cell culture article for a time, as defined herein.

The apparatus, the method of making the apparatus, and the method of using the apparatus, the resulting released cells, and the resulting vessel free of released cells, of the disclosure can include the components or steps listed in the claim, plus other components or steps that do not materially affect the basic and novel properties of the compositions, articles, apparatus, or methods of making and use of the disclosure, such as a particular apparatus configuration, particular additives or ingredients, a particular agent, a particular structural material or component, a particular irradiation condition, or like structure, material, or process variable selected.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The apparatus and methods of the disclosure can include any value or any combination of the values, specific values, more specific values, and preferred values described herein, including explicit or implicit intermediate values and ranges.

In embodiments, the disclosure provides an apparatus and method for removing, for example, cells from a variety of different surfaces and vessel types, individually or in combination, using exposure to and relative motion of an ultrasonic source.

In embodiments, the apparatus and method can, for example:

scan an ultrasonic horn across an exterior surface or plane of a cell culture vessel while the horn is maintained in contact or in close proximity to the vessel;

an exterior surface or a plane of a cell culture vessel can be moved (i.e., scanned) past a stationary ultrasonic horn while the vessel remains in contact with or in close proximity to the horn;

or a combination thereof, such as moving both the horn and the vessel with respect to one another while maintaining contact or close proximity of the vessel surface or plane and the horn.

The disclosure also relates to the use of the disclosed apparatus and method in the release of cells from specialized materials, for example, engineered coatings, shear-thinning polymers, thermally responsive materials, microcarriers, synthetic or native scaffolds, or like materials, and combinations thereof.

The disclosed apparatus and methods can provide advantageous aspects, for example:

significant reductions in time called for to achieve partial or complete cell release from the surface of the culture vessel compared to alternative methods;

significant increases in cell viability, the yield of viable cell released, or both, after the cell release procedure compared to alternative methods;

significant reductions in the energy consumption called for to achieve partial or complete cell release from the surface of the culture vessel compared to alternative methods;

significant reductions in the cost and complexity of the apparatus that can achieve partial or complete cell release from the surface of the culture vessel compared to alternative equipment or apparatus;

a wide variety of laboratory or development cell culture vessels can be used for cell culture and cell release using the disclosed apparatus and method;

the wide variety cell culture vessels can be readily secured to and released from the disclosed cell release apparatus;

potential reduction in the extent and cost of cell purification and concentration operations called for following cell harvest; and like advantages.

Cells have been removed from cell culture surfaces using ultrasonic transducers with a different power coupling method. U.S. Pat. No. 6,143,508, mentions a method of ultrasonic cell removal described in J. Immunol. Methods (1987) 104:1-6, that requires a vessel to be placed in an ultrasonic water bath cleaner. This method of power coupling is less desirable as it risks contaminating the vessel being used and the method takes longer to remove cells from the surface. Another disadvantage is that it can be very cumbersome for use with larger vessels such as Cell Factories, CellSTACK®s, or HYPERStacks®. In contrast the present disclosure provides an apparatus and method that directly couples power against the cell culture vessel's outer wall, and avoids the risk of water bath contamination. The method of the '508 patent can lead to lower cell viability as the cleaning system is designed to induce cavitation. “Alternatively, the cells may be released by physical methods such as mechanical disruption, particularly shearing, such as by vibration, vigorous pipetting, or by sonication using an ultrasonicator and placing the collection device in a water bath,” (see col. 10, line 21).

U.S. Pat. No. 6,086,821 mentions directly coupling an ultrasonic horn to the bottom wall of a multiwell plate that was specially designed with aluminum side walls. The '821 patent also mentions the capability to release particles bound to the vessel surface with this configuration, but the configuration and method was only able to release the particles in a standing wave pattern, thus leaving a majority of particles attached to the surface. The '821 patent also mentions using an ultrasonic transducer and horn to characterize binding forces between molecules by coating particles with one molecule and attaching this molecule to a bound partner molecule on a surface, such as the well of a microplate. The '821 patent mentions measuring the energy required to get the particle to release from the surface. When the ultrasonic horn was contacted with the outside of the well particles could not be removed from the well face in an all polymer plate. With an aluminum well having a plastic bottom, the plastic bottom was contacted with the horn and could only release particles from the surface in a pattern on the well face that is attributable to standing wave formation.

Biomaterials, 33 (2012) 7405-7411, mentions shear stress dependent cell detachment from temperature responsive cell culture surfaces in a microfluidic device. This article provides supporting information about releasing cells from a thermally responsive surface.

Soft Matter, 8 (2012) 260-272, mentions shear-thinning hydrogels for biomedical applications. This article provides background on various shear-thinning materials that can be used in conjunction with a vibration generator to remove cells from growth substrates.

Commonly owned and assigned U.S. Pat. No. 8,114,646, issued Feb. 14, 2012, entitled “Method for Ultrasonic Cell Removal” mentions the use of ultrasonic energy and a horn in a stationary ultrasonic welding set-up to, for example, release biological cells from a stacked culture vessel.

In embodiments, the disclosure provides an apparatus (100) for cell release from a surface of a cell culture article or work piece (101), comprising:

an ultrasonic source (105), e.g., an ultrasonic transducer or an ultrasonic horn and optionally a booster;

a stage comprising a base (112) and a raised portion (114), such as a fence and optionally one or more posts, that defines a cell culture article confinement area; and a mechanism for relative scan motion between the ultrasonic source and the cell culture article confined to the stage.

The mechanism for providing relative scan motion between the ultrasonic source and a cell culture article confined to the stage can be, for example, a race (117), i.e., track or via, and a motive source, e.g., a drive motor (not shown) or like device, wherein the race traverses at least a portion of the long dimension of the cell culture article confined to the stage.

In embodiments, the relative scan motion between the ultrasonic source and a cell culture article confined to the stage can be, for example, linear. In embodiments, the relative scan motion between the ultrasonic source and a cell culture article confined to the stage can be, for example, from about 0.1 to about 3 inches per second, and the ultrasonic source generates ultrasonic waves at a frequency of, for example, from about 16 to about 24 kHz, from about 18 to about 22 kHz, from about 19 to about 21 kHz, and like values, including intermediate values and ranges.

In embodiments, the ultrasonic source can be, for example, an ultrasonic horn emitting at from about 10 to about 80 kHz, and like values, including intermediate values and ranges. The ultrasonic energy generated from just a converter without an ultrasonic horn, i.e., the ultrasonic horn amplifies the signal, can have a frequency, for example, of from about 10 to about 80 kHz. A converter and booster having a horn (i.e., the booster also amplifies the signal) can have a frequency of from about 10 to about 80 kHz. Neither the horn nor the booster changes the frequency. The horn or the booster also does not change the power output. The horn or the booster can increase the amplitude (or stroke length) of the contact face. The horn and booster are both designed to go into matching resonance with the output of the converter. The horn may gain amplitude by metal atoms vibrating in resonance with the converter, which leads to the horn becoming, alternately, physically longer and shorter. The booster is a mechanical design that boosts amplitude by vibrating mechanical parts.

In embodiments, the ultrasonic source can be, for example, an ultrasonic horn emitting at from about 15 to 25 kHz, including intermediate values and ranges.

In embodiments, the cell culture article can be confined to the stage, i.e., the base and the fence combination, and the confinement can be accomplished, for example, without a fastener.

In embodiments, the apparatus can further comprise a mechanism for providing a change in the relative orientation between the ultrasonic source and a cell culture article confined to the stage. In embodiments, there may be no need to rotate the cell culture vessel, i.e., the work piece, such as a roller bottle, since the ultrasonic energy appears to transmit around or through the entire vessel by, for example, contacting the vessel with the horn along a single long axis. A change in the “z” height can be accommodated by raising or lowering the ultrasonic horn, or the vessel. The “x” motion scan (e.g., the scan length of the vessel) can be accomplished by mounting the ultrasonic horn to a slide or race (e.g., a via) that can be driven, for example, pneumatically, electrically, and like motive means, or combinations thereof. The “y” axis can be changed, for example, by moving the fence so as to position all vessels in the same location (i.e., the “y” axis separation between the work piece and the ultrasonic horn. In one example, in the situation of a stationary cell culture article and a scanning ultrasound source, a motor and gripper for rolling a roller bottle, 90, 180, 270, and like incremental angular or rotational degrees, including intermediate angles of rotation can be used. In another example, in the situation of a stationary ultrasound source and a scanning or movable cell culture article, a motor and gripper for rotating the stationary ultrasound source about the article confined to the stage, such as in 90, 180, 270, and like incremental angular or rotational degrees, including intermediate angles of rotation can be used.

In embodiments, the surface of the cell culture article can be, for example, a single layer vessel, a multi-layer vessel, or both, selected from a culture flask, a Petri dish, Multiwell plates, e.g., 384-well plate, a 96-well plate, a 6-well plate, and like vessels, T-75 flasks, T-175 Flasks, T-225 flasks, multilayered flasks, e.g., triple, etc., stacked culture vessels, roller bottles, or combinations thereof, and like vessels, such as HYPERFlasks®, HYPERStacks®, CellSTACK®s, roller bottles, or combinations thereof, including multiples thereof.

In embodiments, the apparatus can further comprise, for example, an optics package in optical communication with the surfaces of the cell culture article.

In embodiments, the apparatus can further comprise, for example, a vibratory energy source, and a thermally responsive material, a shear thinning material, or both, to further increase cell release efficiency or otherwise facilitate processing, during release of cells from the material.

In embodiments, the disclosure provides a method for cell release from a cell culture article, comprising:

securing a cell culture article in the above mentioned cell release apparatus, for example, placing the article against the raised fence and optionally against one or more posts or like members; and

accomplishing relative motion scanning between the energized ultrasonic source and the cell culture article for a time.

In embodiments, the method provides cell release from the surface of the cell culture article, for example, of from about 50% to about 100%, including intermediate values and ranges, after energizing for a time.

In embodiments, the viability of the cells released from the surface of the cell culture article can be, for example, from about 50% to 100%, including intermediate values and ranges.

In embodiments, the cell release method can further include, for example, harvesting the released cells from the cell culture article, and the viability of the harvested cells can be, for example, from about 50% to 100%, including intermediate values and ranges.

In embodiments, the harvested cells can retain all normal functionality compared to enzyme harvested cells.

In embodiments, energizing the ultrasonic source to release cells from the surface of the cell culture article can be, for example, for a time from 0.01 minute to 5 minutes, from 0.05 minute to 2 minutes, from 0.05 minute to 1 minute, including intermediate values and ranges.

In embodiments, the surface of the cell culture article can comprise or can host, for example, a single cell layer, a multi-cell layer, or combinations thereof.

In embodiments, the cell culture article can comprise, for example, a culture flask, a Petri dish, Multiwell plates, e.g., 384-well plate, a 96-well plate, a 6-well plate, etc., T-75 flasks, T-175 Flasks, T-225 flasks, multilayered flasks, e.g., triple, etc., stacked culture vessels, roller bottles, and like vessels, or combinations thereof.

In embodiments, the disclosed apparatus and method can provide superior or unexpected results including, for example, a 2 second scan exposure with the ultrasonic horn released keratinocytes from surfaces of the cell culture article. In comparison a 90 second “high” setting exposure with electromagnetic vibration did not release the keratinocytes from surfaces of the cell culture article.

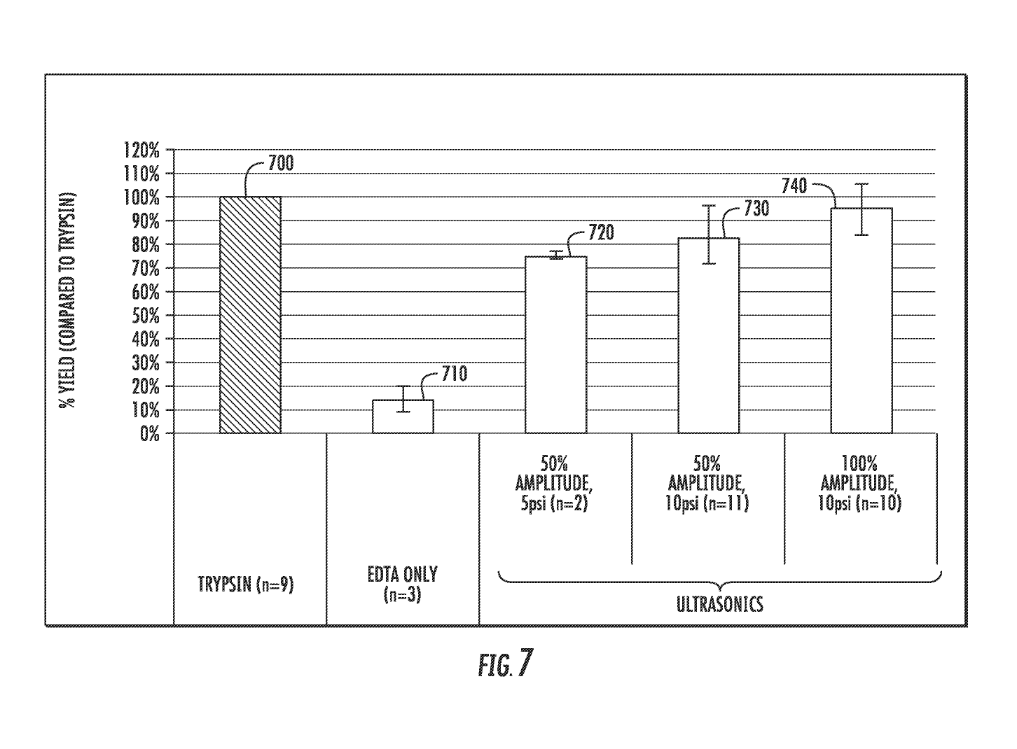

The present disclosure provides an apparatus and method that can remove cells from, for example, an all polymer vessel, and the apparatus and method overcomes the standing wave pattern problem of cell removal using resonant pattern re-localization across the surface of the vessel. A significant aspect of the disclosure is that moving the standing wave resonance pattern by mechanically scanning the location of coupling energy to the vessel while also vibrating at an ultrasonic frequency (preferably at, for example, 20 kHz) such that resonance can occur across the entire surface including near the vessel walls allows for superior cell removal with very short exposure time. Adjusting the amplitude, from about 50% to about 100%, of the ultrasonic horn's vibration (see FIG. 7) and the contact force or clamping force, from about 5 psi to about 10 psi (i.e., the cylinder pressure that was used; the cylinder had a cross sectional area of 0.78 in², which translates into a contact force of about 3.9 pounds force to about 7.8 pounds force, allows for complete removal of high viability cells in a cell culture vessel. Mechanically scanning the layers across vessels as large as a 40 layer CellSTACK®s or a HYPERStack®-120 is possible with a multi-axis actuator, allowing the present disclosure to remove cells on all common commercial adherent cell culture vessels.

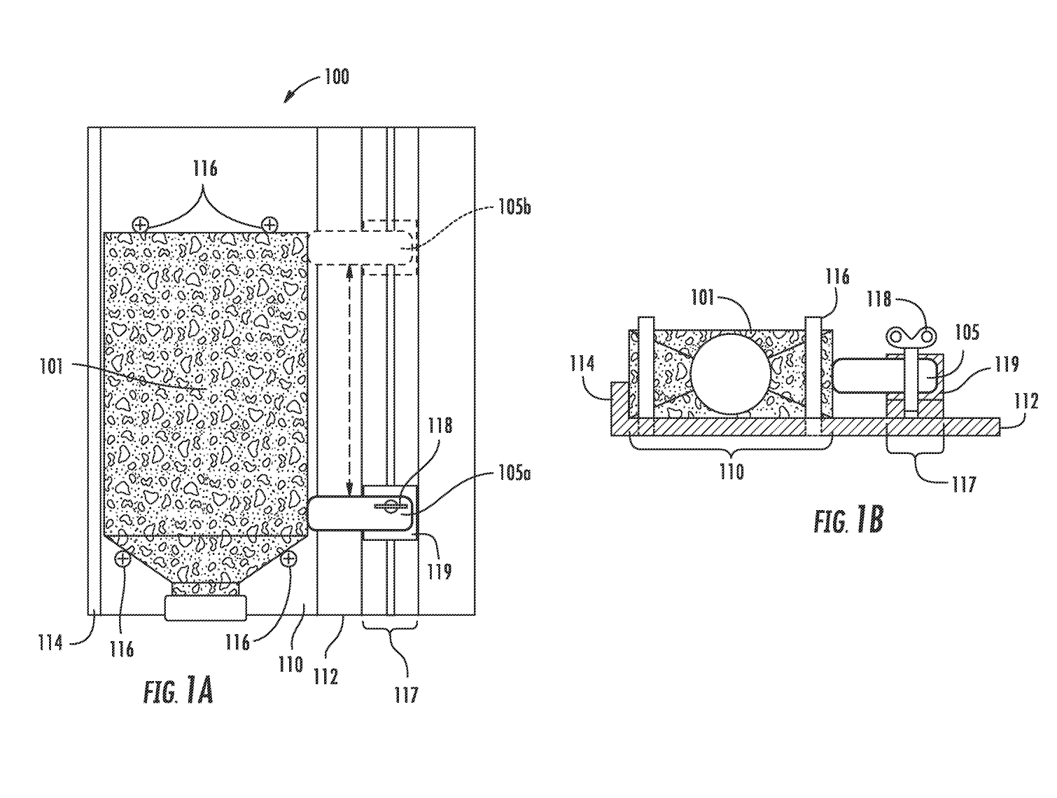

Referring to the Figures, FIGS. 1A and 1B shows, respectively, top and side views of an exemplary apparatus having a movable ultrasonic source supported on a race structure adjacent to a retained and supported culture vessel. FIG. 1A shows a top view of the apparatus (100) having an ultrasonic horn (105) supported on a race structure (117), which structure is adjacent to a nested or retained culture vessel(s) (101) on a stage (112). In embodiments, the vessel(s) (101) can be supported by a base (110) or optionally further elevated (not shown). The race structure (117) and associated retainer support (119) and fastener (118) such as a thumb screw (shown), enables scanning the horn over the entire or a portion of the length of a cell culture vessel (e.g., initial position 105a to final position 105b). The culture vessel (e.g., HYPERFlask®-vessel shown) can be passively held in place by a raised portion or fence (114) situated on a side of the vessel opposite the horn, and one or more optional posts (116). The fence and post positions can be readily changed or reconfigured as desired or called for based on the dimensions of the culture vessels to be retained in the retention area of nest of stage (112). FIG. 1B shows the side view of the apparatus (100) of FIG. 1A having the ultrasonic horn (105) supported on the race structure (117) having, for example, a retainer support (119) and fastener (118), which structure is adjacent to the nested or retained culture vessel(s) (101) on the stage (112) and base (110).

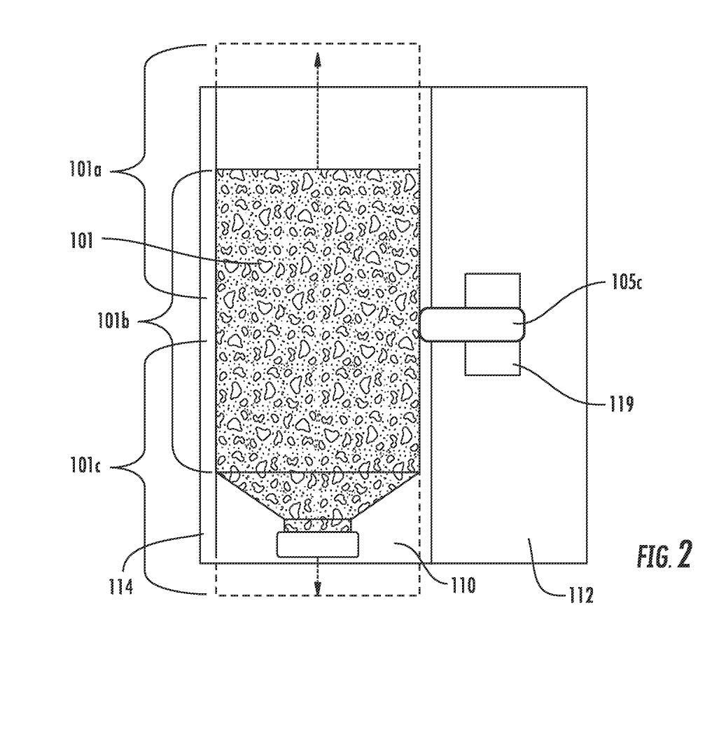

FIG. 2 shows a top view of an alternative configuration of the apparatus of FIG. 1 having, instead of a scanning ultrasonic horn, a stationary ultrasonic horn (105c) and retainer support (119) attached to the stage (110), and a movable structure (not shown, e.g., a push rod and motor or a conveyor belt) for moving the vessel, for example, moving the vessel (101) itself, moving the stage (112), moving the base (110), or moving combinations thereof with respect to the stationary ultrasonic horn (105). In embodiments, the vessel (101) can be initially positioned or offset (101a) with respect to the stationary horn (105c). In embodiments, the vessel (101) can be advanced or moved, sequentially or continuously, to another intermediate position (101b), by any suitable motive force, such as a servo motor or convey belt. In embodiments, the vessel (101) can be further advanced or moved, sequentially or continuously, to yet another and final position (101c). In embodiments, the vessel (101) can be retained between just the fence (114) and the stationary horn (105c), or alternatively or additionally with one or more posts (116) (not shown).

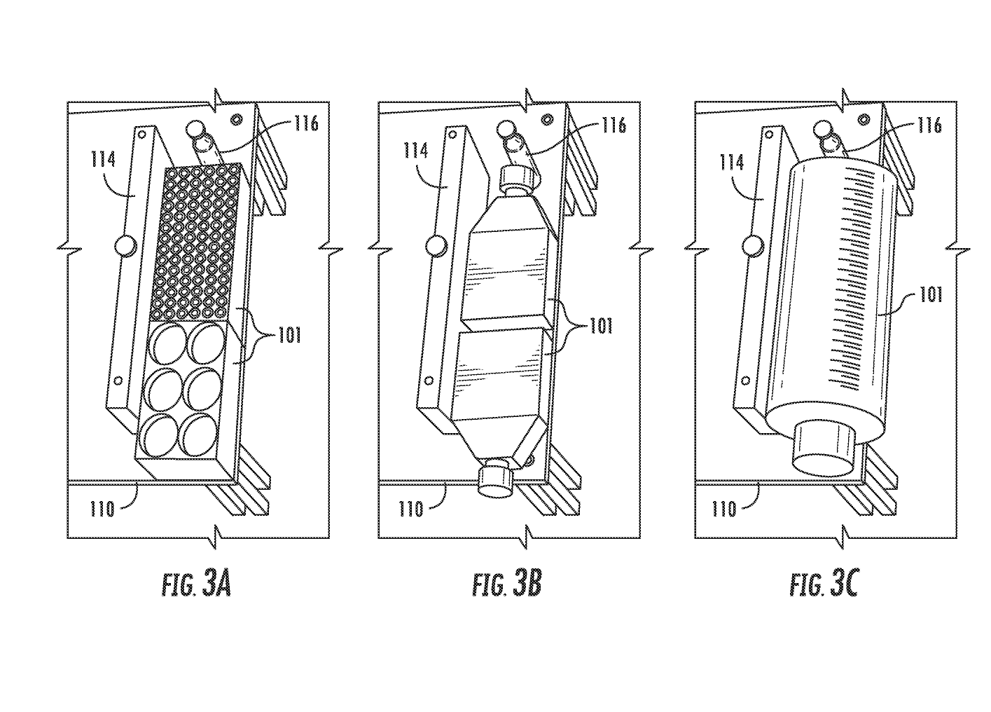

In embodiments, the disclosed apparatus and method of use can feature simple nesting of the cell culture vessel, that is in a retention arrangement for the vessel (i.e., work piece), that can accommodate one or more culture vessel, such as multiple culture vessels, different culture vessel types, different culture vessel geometries, and like vessel structural variations, such as illustrated in FIGS. 3A to 3C. FIGS. 3A to 3C show images of useful exemplary alternative cell culture vessel types nested in the disclosed apparatus. FIG. 3A shows two multiwell plates (101) in the retention area (i.e., nest) situated end-to-end. In embodiments, two additional multiwell plates shown, and like multiples, can be situated atop the two multiwell plates already situated in the nest (not shown). In embodiments, the horn or a plurality of horns (not shown) can scan an individual vessel or a multiplicity of stacked vessels, for example, sequentially or simultaneously, with for example, suitable adaptation for vertical displacement of the horn(s) or the vessel(s). FIG. 3B shows two T-75 flasks (101) in the nest also situated end-to-end. In embodiments, two additional T-75 flasks (not shown), or like flasks, can be situated atop the two T-75 flasks already situated in the nest. FIG. 3C shows a roller bottle held in place by the same or similar fence (114) and post structures (116). The vessels shown were demonstrated to have similar multimodal patterning over their entire cell culture surfaces when exposed to relative motion of the ultrasonic horn (not shown) or multiple horns. The standing wave patterns generated within each of the different cell culture vessels when exposed to the ultrasound were shifted when the active horn was scanned along a side of the vessel.

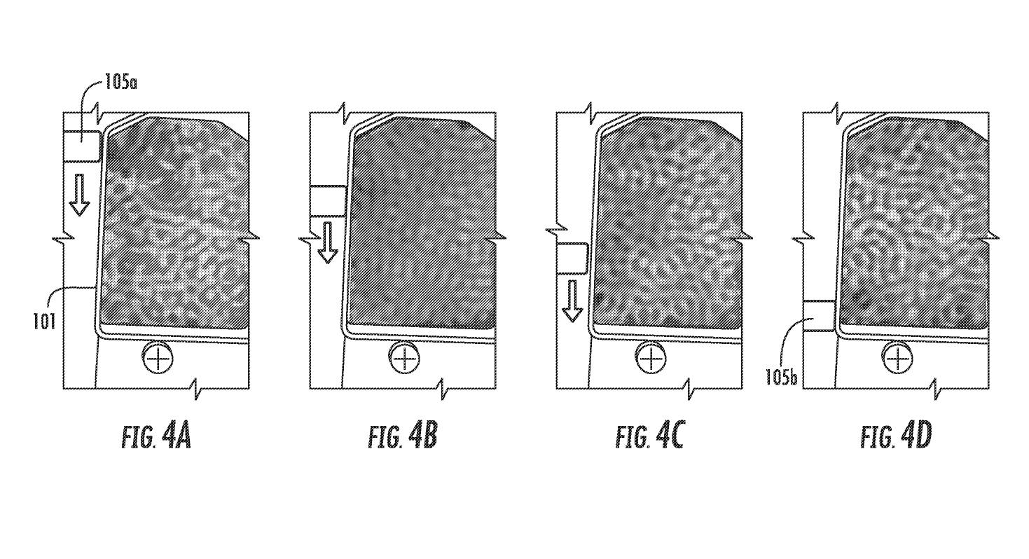

FIGS. 4A to 4D show an exemplary top view imaging sequence obtained from scanning the length of a stationary cell culture vessel (101) with a movable 20 kHz horn (105). In embodiments, the horn is initially positioned (105a) and scans along the left side of the vessel to a final position (105b). As the horn passes along the vessel antinode patterns shift to allow regions of resonance to ultimately cover all areas of the cell culture surface and effectively results in releasing substantially all cells that adhere to the vessel surface.

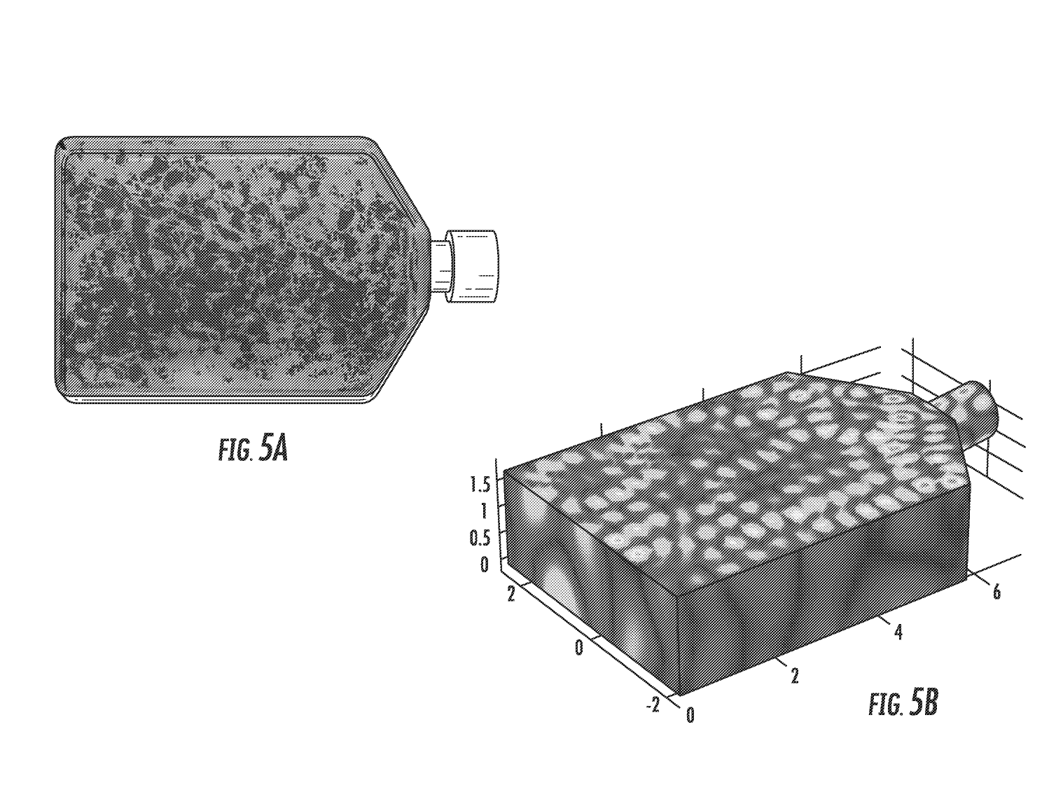

FIGS. 5A and 5B show examples of actual and modeled cell release patterns, respectively. FIG. 5A show an example of the cells that remain attached to the cell culture surface and appear as a pattern of nodes (dark regions), that is areas without resonant agitation, and are distinctly stained with crystal violet unlike the resonantly agitated areas (light regions). FIG. 5B shows an example of a Comsol model having 20 kHz of ultrasonic horn energy input on the surface of a T-175 vessel. If the ultrasonic horn remains stationary, that is not scanned, cells are only released from areas where resonating regions are located (i.e., light regions that represent antinode containing “islands” in a dark colored nodal zone “sea” that is not resonating).

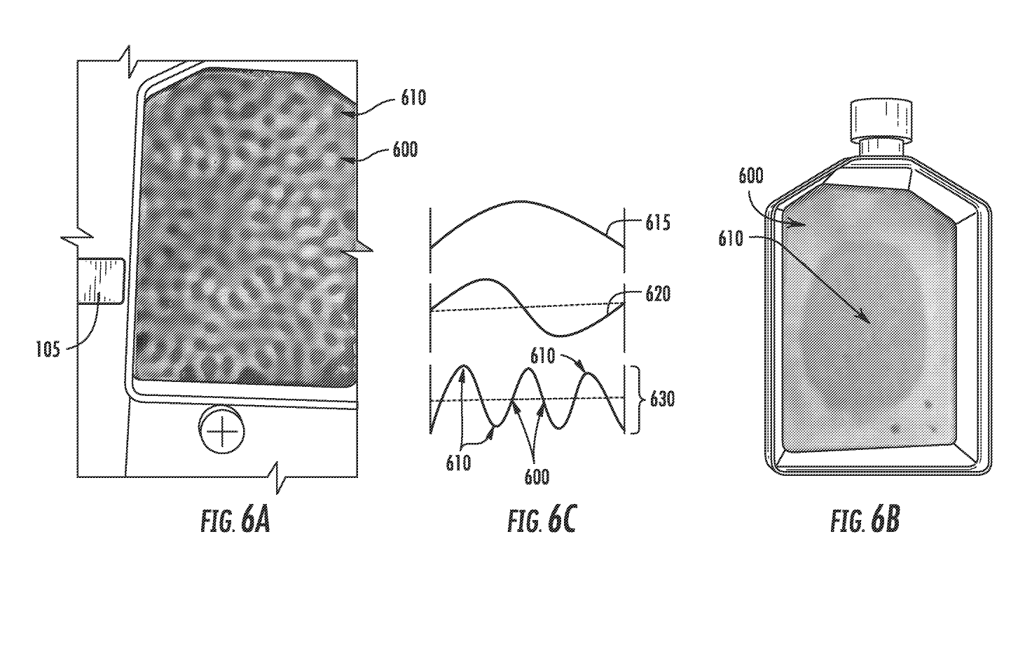

FIGS. 6A and 6B show, respectively, examples images of modal patterns visualized with crystal violet stain. FIG. 6A shows an example of a multimodal pattern in a cell culture vessel (T-175 flask) being ultrasonicated at 20 kHz. The stain has organized into a multimodal pattern of nodes (600) (light) and antinodes (610) (dark), with the stain accumulating at the resonating zones containing the antinodes. The antinodes are the center of the regions of resonant energy that release cells from the culture surface. FIG. 6B shows an example demonstrating a single node (600) a single antinode (610) at 300 Hz generated with a comparative electromagnetic shaker (EMS) device. FIG. 6C shows examples of modal (mode 1 (615); mode 2 (620)) and multimodal (630) (i.e., as achieved in the present disclosure) waveforms that graphically illustrate vibrational nodes (600) and antinodes (610).

As seen in FIG. 6A, a 20 kHz ultrasonic system creates a complex standing wave pattern across the cell culture surface of the vessel. Of particular interest is the ability of ultrasonic systems to generate resonance in the vessel surface near the walls of the vessel, where the vessel has high sectional modulus. This high sectional modulus limits the percentage of surface area that lower frequency vibrational systems can resonate with sufficient acceleration to release cells (FIG. 6B). Unfortunately, at 20 kHz large areas of the surface act as nodes between the resonating regions (see FIG. 5). In the present disclosure this limitation is overcome by scanning the horn across the vessel, or moving the vessel with respect to the horn, to change the coupling point and thus the standing wave pattern. This allows the resonating islands on the cell culture surface to change locations as seen in, for example, FIGS. 4A to 4D. The zones of resonance are thus able to move across the cell culture surface, allowing virtually the entire surface to experience accelerations that generate forces sufficient to release cells.

Although not limited by theory, if the ultrasonic horn of the apparatus is fixed in a single location on the surface of a cell culture vessel, a stable standing wave multimodal pattern develops. Antinodes mark the centers of regions of resonant energy that generate the lateral displacement and acceleration sufficient to produce the shear force required to release cells for the vessel surface. However, not all locations (i.e., everywhere) on the surface of the cell culture article are covered by or included in these antinode resonating zones. By scanning, for example, passing or moving, the horn (105) along a plane of the culture vessel (101), antinode positions shift and most of the cell culture surface area is exposed to the shear force called for to remove all cells. Additionally, nesting for the cell culture vessel can include, for example, a simple fence and a post. In a comparative electromagnetic shaker (EMS) apparatus and method, a single frequency results in a single node and single antinode (e.g., FIG. 6B). To agitate more of the cell culture surface area, different frequencies must be used to cause the antinode to move around the cell culture surface to release cells. The nesting for the EMS is more complex, for example, having clamps and like active retainers, that call for, for example, considerably greater operator manipulation. In contrast, the disclosed apparatus includes an ultrasonic nest and ultrasonic energy generator combination that have a considerably simplified structure. In embodiments, the disclosed apparatus and method can be used, for example, alone or as an adjunct to other methods for releasing cells from surfaces, such as shear-thinning or thermally responsive materials.

FIG. 7 illustrates that variation of parameters can provide improved and maximized yields of cell release. Parameters can be changed to accomplish the goals of high cell viability and high cell yield. The bar (700) indicates the percentage of CHO K1 cells harvested by an enzymatic (trypsin) methods, and is normalized to 100%. In the present disclosure, the procedure for mechanical cell release can include rinsing the cells with EDTA (a calcium and magnesium chelator) prior to placing the culture vessel in the cell release equipment. The EDTA only bar (710) indicates the number of cells removed with just EDTA (i.e., no mechanical agitation), was about 15%. Bars (720), (730), and (740), respectively, indicate the percentage of cells removed with EDTA treatment followed by agitation using varied ultrasonic equipment parameters: 50% amplitude and 5 psi horn contact force of the cylinder (25 mm bore diameter), i.e., pressing the cylindrical horn against the culture vessel, yielded about 75% release of cells from the culture vessel (720); 50% amplitude and 10 psi yielded about 85% release of the cells from the culture vessel (730); and 100% amplitude and 10 psi yielded about 95% release of the cells from the culture vessel (740). The scan speed in each of these examples was 2 inches per second and the overall ultrasonic exposure time was 2.5 seconds. This scan speed can also be varied to accomplish improved cell release. All cell viabilities were greater than 90% (data not shown). The cell culture vessels used in this experiment were HYPERFlasks. FIG. 7 demonstrates that the force to release cells can be optimized by the amplitude of the resonating ultrasonic horn and the force (e.g., contact or clamping force) at which the horn is pressed against the cell culture vessel.

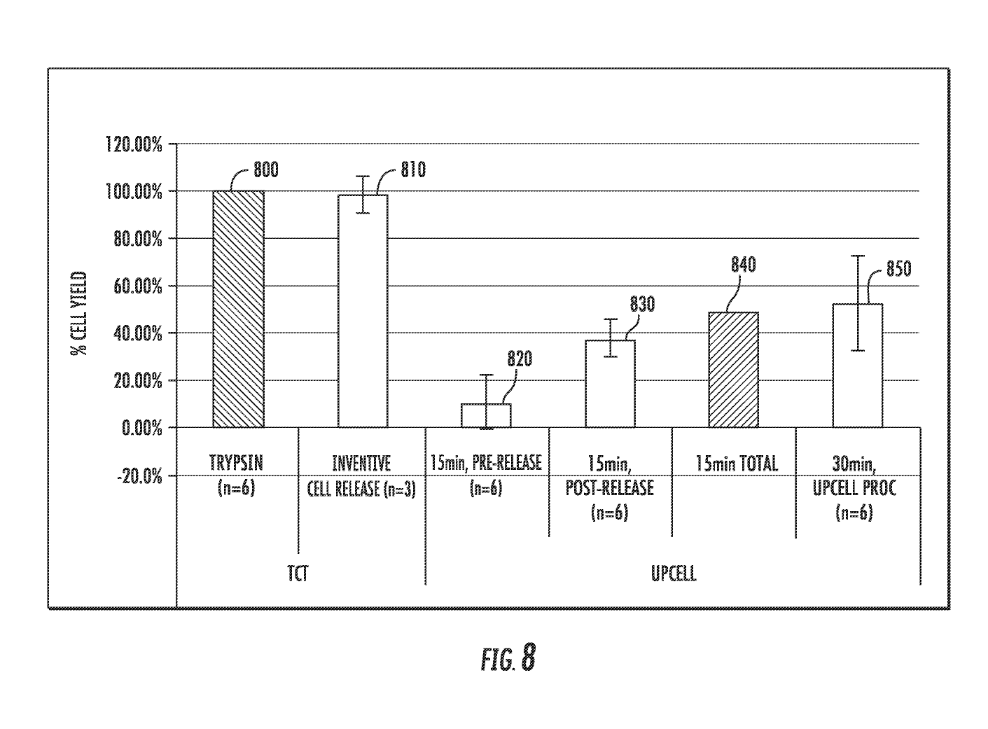

FIG. 8 shows a bar chart of CHO K1 Cell yield on UPCELL™ (Nunc) plates with and without use of the disclosed apparatus and method for cell release. These plates were compared to tissue culture-treated (TCT) 6-well plates seeded with the same number of cells as Nunc UPCELL™ 6-well plates. The TCT plates were harvested with trypsin according to standard protocol (bar 800), or with the disclosed apparatus and method for cell release “inventive cell release” (bar 810). The disclosed apparatus and method for cell release were used for harvesting the TCT plates as follows. First the culture medium was removed and the residual cells were rinsed with 0.5 mM EDTA/0.1% pluronic in calcium and magnesium-free phosphate buffered saline (dPBS), then the cells in the vessel were subjected to ultrasonic exposure and scanning FIG. 8 demonstrates the usefulness of the inventive cell release apparatus and method in conjunction with a thermally responsive polymer. UPCELL™ is a commercially available thermally responsive product sold by Nunc. The thermally responsive polymer is an n-isopropyl acrylamide derivative that has a lower critical solution temperature of 30° C. A conformational change occurs in the polymer above 30° C. to make it hydrophobic to bind cells, and below 30 degrees to make it hydrophilic and release cells. The results disclosed herein appear to indicate that the transition is not complete after 30 minutes at 23° C., since the cell yield was only 52.7% of the number of cells harvested from the TCT plate by trypsin (trypsin control harvest is considered to achieve 100% yield). Incubating an UPCELL™ plate at room temperature (at about 23° C.) for 15 minutes, and then augmenting the thermal release polymer with the disclosed ultrasonic cell release method of 20 kHz, 50% amplitude for 1.7 seconds, enabled release of the same percentage of cells released by the thermal release polymer alone after 30 minutes. The disclosed ultrasonic cell release apparatus and method effectively decreased processing time by, for example, 50% or more. Similar to the effect in FIG. 8, is the effect obtained with cells growing on a shear-thinning polymer or hydrogel such as locust bean gum. While the polymer is stationary, the functional groups of the polymer network can self-assemble and form a gel to which cells can adhere. If the polymer is subjected to motion, the interactions between the functional groups of the polymer network are interrupted. This causes the polymer network to take on a fluid consistency that can release the cells from the surface. Examples of shear-thinning materials include, for example, polymer blends such as hyaluronic acid and methylcellulose, colloidal gels such as poly(D,L-lactic-co-glycolic acid), and block co-polymers such as a cyclodextrin in combination with poly(ethylene oxide), other protein and peptide hydrogels, and like materials (see also Soft Matter mentioned supra.).

Referring further to FIG. 8, UPCELL™ plates were pre-coated having the above mentioned thermally responsive acrylamide polymer, which can have a critical solution temperature of about 30° C. Above 30° C., the polymer conformation is hydrophobic, and cells adhere to the coated surface. Below 30° C., the polymer changes conformation to become hydrophilic and supposedly releases the cells. The UPCELL™ plates including cell culture were removed from an incubator and placed on the bench top at room temperature (23° C.). After 15 minutes, the medium was removed from one of the plates and the number of cells in the medium were counted, i.e., pre-ultrasonication cell count or pre-release (820). The plate was then subjected to the disclosed scanning ultrasonic cell release method. The released cells were collected in cell culture medium and counted. The number of cells from the UPCELL™ plate collected after the disclosed scanning ultrasonic cell release method (37.9%) (post-cell release 830) was more than three times the amount of cells released by UPCELL™ alone after 15 minutes (11%)(pre-cell release 820). The amount of cells collected from combining these volumes (i.e., stripped bar 840=bar 820+bar 830) was equivalent to the total number of cells collected from the UPCELL™ plates that were harvested after 30 minutes without application of the disclosed ultrasonic cell release method (bar 850). When the disclosed cell release method was used the cell release processing time was halved or, for example, reduced by 15 minutes.

In embodiments, the disclosed apparatus and method can be, for example, automated and used for cell release on any desirable scale, such as on a laboratory scale, on a pilot scale, or on a large scale industrial production, including intermediate scales. In embodiments, a plurality of HYPERStack® cell culture vessels situated on, for example, a suitable shelving unit can be exposed to or irradiated by a moving ultrasonic source to achieve efficient cell release. An ultrasonic horn source can be, for example, mounted on a drive belt that scans multiple vessels on one or more levels in an automated fashion. Alternatively, the HYPERStack® cell culture vessels situated on, for example, a shelving unit can be moved past a stationary ultrasonic source, for example, in a conveyor belt fashion to achieve efficient cell release.

To keep the vessel from moving during the mechanical scan with a moving ultrasonic source a post (116) can be used in addition to the fence (114) as shown in FIGS. 1 and 3. Other means to nest the vessel can be used to ensure the vessel maintains its location as the horn is scanned across the vessel. An option for a fully automated system can involve, for example, maintaining cell culture vessels on a standard shelving unit constructed with fences and posts for each vessel or stacked group of vessels, and placed in a incubating room. After exposing the cells in culture to a cell release solution (such as EDTA) for the appropriate length of time (based on the adherent properties of the cells to the particular substrate), an ultrasonic system on a belt drive can make contact with the vessels on the shelving unit according to programmable features in the drive unit, for the purpose of releasing cells from many vessels in series, and or in parallel.

In embodiments, the ultrasonic vibrational energy can be coupled to a cell culture vessel by simply contacting or pressing a vibrating metal face of the ultrasonic source against a cell culture vessel. In embodiments, the cell culture vessel can be pressed against the backstop wall or the fence by the ultrasonic source. Alternatively or additionally, the cell culture vessel can be independently secured against the backstop wall or the fence to provide further immovability to vessel. The contacting face of the ultrasonic source can vibrate by way of an ultrasonic transducer that can consist of, for example, a piezo electromechanical converting unit (the converter) that is driven by voltage and modulated via a wave generating power supply. Examples of suitable frequencies can be, for example, from 10 to 90 kHz, including intermediate values and ranges. A preferred operating frequency can be, for example, around or about 15 to 25 kHz, and more preferably about 20 kHz. While the face of the converter can be used to contact the cell culture vessel, the contact vibrating face can more preferably be in the form of an ultrasonic horn that vibrates in resonance with the output of the converter, for example, vibrating also at preferably 20 kHz. The face of the converter can be selected or shaped to provide maximum contact with the vessel being ultrasonicated and to increase the amplitude of vibration.

The disclosure has been described with reference to various specific embodiments and techniques. However, it should be understood that many variations and modifications are possible while remaining within the scope of the disclosure.

What is claimed is:

1. An apparatus for cell release from a surface of a cell culture article, comprising:
an ultrasonic source;
a stage comprising a base and a raised portion that defines a cell culture article confinement area; and
a mechanism for relative scan motion between the ultrasonic source and the cell culture article confined to the stage.

2. The apparatus of claim 1 wherein the mechanism for providing relative scan motion between the ultrasonic source and a cell culture article confined to the stage comprises: a race and a motive source, wherein the race traverses at least a portion of the long dimension of the cell culture article confined to the stage.

3. The apparatus of claim 1 wherein the relative scan motion between the ultrasonic source and a cell culture article confined to the stage is linear and continuous.

4. The apparatus of claim 1 wherein the relative scan motion between the ultrasonic source and a cell culture article confined to the stage is from about 0.1 to about 3 inches per second, and the ultrasonic source generates ultrasonic waves at a frequency from about 19 to about 21 kHz.

5. The apparatus of claim 1 wherein the ultrasonic source is an ultrasonic horn emitting at from about 10 to about 80 kHz.

6. The apparatus of claim 1 wherein the ultrasonic source is an ultrasonic horn emitting at from about 15 to 25 kHz.

7. The apparatus of claim 1 wherein the cell culture article confined to the stage is accomplished without a fastener.

8. The apparatus of claim 1 further comprising a mechanism for providing a change in the relative orientation, position, or both, between the ultrasonic source and a cell culture article confined to the stage.

9. The apparatus of claim 1 wherein the surface of the cell culture article comprises a single layer vessel, a multi-layer vessel, or both, selected from vessels comprising at least one of a culture flask, a Petri dish, Multiwell plates, T-75 flasks, T-175 Flasks, T-225 flasks, multilayered flasks, stacked culture vessels, roller bottles, or combinations thereof.

10. The apparatus of claim 1 further comprising an optics package in optical communication with the surfaces of the cell culture article.

11. A method for cell release from a cell culture article, comprising:
securing a cell culture article in the cell release apparatus of claim 1; and
accomplishing relative motion scanning between the energized ultrasonic source and the cell culture article for a time.

12. The method of claim 11, wherein 50% to 100% of cells are released from the surface of the cell culture article after energizing and scanning at 100% ultrasonic amplitude and 10 psi contact force for a time.

13. The method of claim 11 wherein the viability of the cells released from the surface of the cell culture article is from about 50% to 100%.

14. The method of claim 11 further comprising harvesting the released cells from the cell culture article, wherein the viability of the harvested cells is from about 50% to 100%.

15. The method of claim 14 wherein the harvested cells retain substantially all normal functionality compared to enzyme harvested cells.

16. The method of claim 11 wherein scanning is for a time of from about 0.01 minute to about 5 minutes.

17. The method of claim 11 wherein scanning is for a time of from about 0.05 minute to about 2 minutes.

18. The method of claim 11 wherein the surface of the cell culture article comprises a single cell layer, a multi-cell layer vessel, or combinations thereof.

19. The method of claim 11 wherein the cell culture article comprises a vessel selected from at least one of a culture flask, a Petri dish, Multiwell plates, T-75 flasks, T-175 Flasks, T-225 flasks, multilayered flasks, stacked culture vessels, roller bottles, or combinations thereof.

20. The method of claim 11 wherein the cell culture article comprises at least one surface comprising a thermally responsive material, a shear thinning material, or a combination thereof.
